(19) United States
(12) Patent Application Publication
(10) Pub. No.: US 2005/0164373 A1
(43) Pub. Date: Jul. 28, 2005

(54) Diffusion-aided loading system for microfluidic devices

(76) Inventors: Mark F. Oldham, Los Gatos, CA (US); Ian A. Harding, San Mateo, CA (US); Kevin S. Bodner, Belmont, CA (US)

Correspondence Address: KILYK & BOWERSOX, P.L.L.C., 3603 CHAIN BRIDGE ROAD, SUITE E, FAIRFAX, VA, 22030, US

(21) Appl. No.: 10/762,786

(22) Filed: Jan. 22, 2004

(51) Int. Cl. C12Q 1/68; C12M 1/34

(52) U.S. Cl. 435/287.2; 366/341

(57) ABSTRACT

Microfluidic devices having a diffusion-aided system for loading samples into the microfluidic device are provided. Methods of gas-venting a microfluidic device through a non-porous, gas permeable material sealing cover layer, for example, during liquid sample loading, are also provided. The non-porous, gas-permeable material can be, for example, a polysiloxane, for example, polydimethylsiloxane.

### FIELD

The present application relates to microfluidic devices, systems that include such devices, and methods that use such devices and systems.

### BACKGROUND

Microfluidic devices are used for manipulating fluid samples. There continues to be a need for methods and microfluidic devices that are capable of efficiently and effectively providing very large numbers of small volume samples or sample portions in discrete sample-containing wells. There continues to exist a demand for microfluidic devices that develop less back pressure than conventional microfluidic devices when filled with a liquid sample, and that provide less trapped air in the device than conventional microfluidic devices, upon being filled with a liquid sample.

### SUMMARY

According to various embodiments, a microfluidic device is provided that includes at least one sample-containment region, a non-porous, gas-permeable material sealing device at least partially defining the at least one sample-containment region, and an input opening in fluid communication with the at least one sample-containment region. The sealing device can be, for example, a plug or a cover layer such as a sheet or a strip. The non-porous, gas-permeable material can be, for example, a polysiloxane material.

According to various embodiments, a microfluidic device is provided and includes at least one sample-containment region, at least one venting region, at least one non-porous, gas-permeable sealing device that at least partially defines the at least one venting region and that includes a non-porous gas-permeable material having a permeability coefficient relative to oxygen gas (O₂) of at least about 8×10¹⁵ at about 35° C., and at least one non-gas-permeable material at least partially defining the at least one sample-containment region.

According to various embodiments, a method for venting a gas from a microfluidic device is provided that includes loading a liquid-containing sample into a microfluidic device, and venting a gas from the microfluidic device through a non-porous, gas-permeable material sealing device. The sealing device can at least partially define a sample-containment region of the microfluidic device, at least partially define a venting region of the microfluidic device, or at least partially define both such regions. The sealing device can be in the form of, for example, a plug or a cover layer, for example, a sheet or a strip. The non-porous, gas-permeable material can be, for example, a polysiloxane material.

### BRIEF DESCRIPTION OF THE DRAWINGS

Various embodiments of the present teachings are exemplified in the accompanying drawings. The teachings are not limited to the embodiments depicted, and include equivalent structures and methods as set forth in the following description and known to those of ordinary skill in the art. In the drawings:

FIG. 1 is a perspective view of a microfluidic device in an unassembled state according to various embodiments and including a hinged substrate support;

FIG. 2 is an enlarged cross-sectional side view of the microfluidic device similar to that shown in FIG. 1 but including a greater number of sample-containment regions, shown in an assembled state, and including non-porous, gas-permeable material sealing plugs and a non-porous, gas permeable material cover layer in the form of a substrate support 22;

FIG. 3 is a perspective exploded view of a substrate support for a microfluidic device and a mask utilized in a method of preparing the microfluidic device, according to various embodiments;

FIG. 4 is an enlarged view of a section of the substrate support shown in FIG. 3;

FIG. 5 is a perspective view of a microfluidic device according to various embodiments and including a sealing cover layer in the form of a sheet;

FIG. 6 is a plan view of a microfluidic device according to various embodiments and including a venting region; and

FIG. 7 is a perspective view of a vacuum aided loading system for a microfluidic device according to various embodiments.

It is to be understood that both the foregoing general description and the following detailed description are exemplary and explanatory only and are intended to provide a further explanation of the various embodiments of the present teachings.

### DETAILED DESCRIPTION OF CERTAIN EMBODIMENTS

According to various embodiments, a microfluidic device is provided that can include: at least one sample-containment region capable of containing a sample; a non-porous, gas-permeable material sealing device; an input opening in fluid communication with the sample-containment region; and an optional venting region. The non-porous, gas-permeable sealing device can vent a gas from the sample-containment region or from the optional venting region, for example, the device can vent gas displaced from the sample-containment region during a liquid sample loading procedure. The non-porous, gas-permeable material sealing device can include a material that permits molecular diffusion of a gas therethrough while providing a liquid barrier that prevents diffusion of aqueous samples, for example, at water pressures of up to about 50 psi and at a temperature from about 25° C. to about 95° C. The non-porous, gas-permeable material can be, for example, a hydrophobic material. The non-porous, gas-permeable material can be, for example, a hydrophilic material. The non-porous, gas-permeable material can be, for example, a polysiloxane material.

According to various embodiments, the non-porous, gas-permeable material can be a material that, when used as a sealing member for a microfluidic device, provides a sufficient amount of gas permeability, as defined by a minimum O₂ permeability coefficient. The permeability coefficient (“P”), as set forth in “Permeability and Diffusion Data” by S. Pauly, pp. VI/543-569 in Polymer Handbook, 4^{th} Ed., Wiley-Europe (1999) (“Pauly”), the complete disclosure of which is hereby incorporated by reference in its entirety for all purposes, and is defined as P=(quantity of permeant) (film thickness). (area) (time) (pressure drop across film)

As set forth by Pauly, the permeability coefficient is not only a function of the chemical structure of the polymer; it also varies with the morphology of the polymer and depends on many physical factors such as density, crystallinity, and orientation. However, the chemical structure of a polymer can be considered to be the predominant factor which controls the magnitude of the permeability coefficient.

According to various embodiments, the minimum permeability coefficient of the non-porous, gas-permeable material for oxygen gas (O₂) at 35° C. can be at least about 8×10¹⁵, for example, at least about 24×10¹⁵, at least about 165×10¹⁵, at least about 250×10¹⁵, at least about 350×10¹⁵, at least about 500×10¹⁵, or at least about 690×10¹⁵. The permeability coefficients for various polysiloxanes are set forth in Table 1.11 in Pauly.

According to various embodiments, the non-porous, gas-permeable material can be a material that, when used as a sealing member for a microfluidic device, provides a sufficiently low amount of porosity. As used herein, “non-porous” refers to material that does not permit gas transport through through-hole pores but rather restricts gas transport to molecular diffusion through the non-porous material.

According to various embodiments, the sealing device can be in the form of a sealing plug or a sealing cover layer such as a film, sheet, or strip.

According to various embodiments, the microfluidic device can include: at least one sample-containment region capable of containing a sample; an input opening in liquid fluid communication with the sample-containment region; an outlet opening in liquid fluid communication with the sample-containment region; and a non-porous, gas-permeable material sealing plug disposed within the outlet opening. The non-porous, gas-permeable material sealing plug can allow gas to molecularly diffuse therethrough while providing a barrier against the escape of liquid from the sample-containment region. The plug can be located in, on, or over the sample-containment region. Gas that can be vented through the sealing plug can include, for example, gas displaced from a liquid sample loading procedure. The non-porous, gas-permeable material sealing plug can include a hydrophobic material that permits molecular diffusion of a gas therethrough while providing a liquid barrier that prevents diffusion of aqueous samples, for example, at water pressures of up to about 50 psi at 95° C. The non-porous, gas-permeable material can include, for example, a hydrophobic material. The non-porous, gas-permeable material can instead or additionally include, for example, a hydrophilic material. The non-porous, gas-permeable material can be, for example, a polysiloxane material.

According to various embodiments, the microfluidic device can include at least one reaction site; at least one input opening in liquid fluid communication with the at least one reaction site; and at least one channel connecting the at least one reaction site with the at least one input opening. According to various embodiments, the microfluidic device can include a plurality of reaction sites; a plurality of input openings; and a plurality of channels connecting at least one of the plurality of reaction sites with at least one of the plurality of input openings.

According to various embodiments, the microfluidic device can include nucleic acid sequence probes and/or nucleic acid sequence primers to perform PCR that have been deposited in one or more of the sample-containment regions present in the microfluidic device. Deposition of such probes and primers can be accomplished, for example, by a drying down technique. A reaction mixture including, for example, magnesium salts, nucleic acid sequence bases, and a sample containing an analyte of interest can be added to the microfluidic device containing the formerly deposited nucleic acid sequence probes and/or nucleic acid sequence primers. A PCR reaction can then be conducted using the prepared microfluidic device. Alternatively, a sample containing an analyte of interest can be deposited in the sample-containment regions of the microfluidic device, followed by addition of a reaction mixture including, for example, nucleic acid sequence probes and nucleic acid sequence primers, magnesium salts, and nucleic acid sequence bases.

According to various embodiments, the microfluidic device can include at least one sample-containment region containing a sample or substance disposed therein. The substance can include a nucleic acid sequence probe, a nucleic acid sequence primer, a sample containing an analyte of interest, or another substance of interest, any of which can be in a solid form, a liquid form, or in a dried form. According to various embodiments, the microfluidic device can include a sample or substance disposed in each one of the sample-containment regions of the device, or only in selected sample-containment regions. According to various embodiments, the sample can be disposed in selected columns or selected rows of the array of the sample-containment regions of the microfluidic device. According to various embodiments, the microfluidic device can include valving to permit deposition of a sample or reagent to any number of combinations of the sample-containment regions of the microfluidic device.

According to various embodiments, the sample-containment regions of the microfluidic device can be completely filled with sample or reagent with any trapped gas diffusing through the non-porous, gas-permeable material cover layer. By providing completely filled sample-containment regions the occurrence of condensation can be decreased, thereby decreasing the occurrence of loss of optical signal due to condensation in the optical path.

According to various embodiments, the above described prepared microfluidic devices can include a non-porous, gas-permeable material sealing at least the input openings and outlet openings of the microfluidic device to allow gas to molecularly diffuse therethrough while providing a barrier against the escape of liquid from the sample-containment regions.

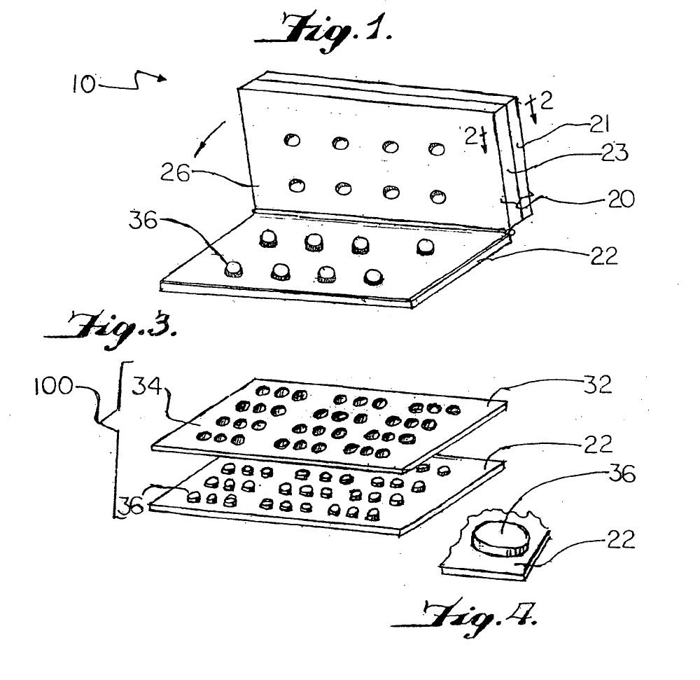

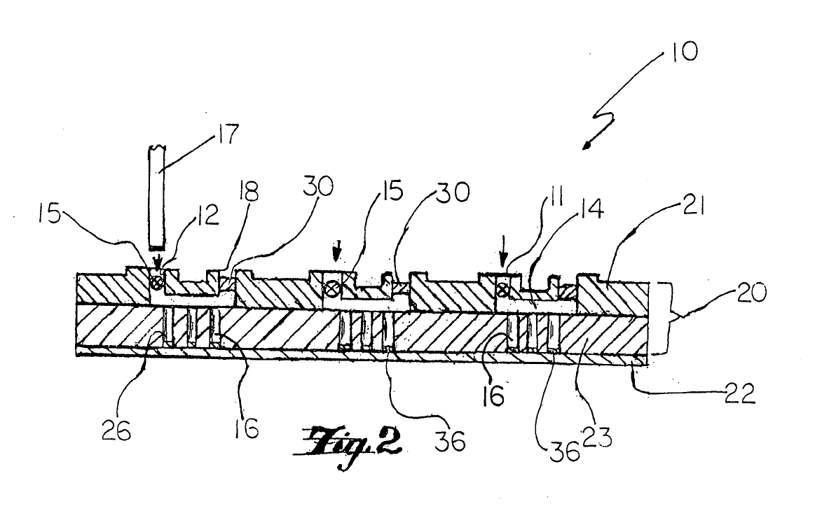

FIG. 1 is a perspective view of an embodiment of a microfluidic device 10 that includes a substrate 20 and a non-porous, gas-permeable material sealing cover layer in the form of a substrate support 22. The substrate 20 can include a plurality of input openings, input channels, sample-containment regions, and outlet openings, for example, as shown in FIG. 2 and described below. The substrate 20 can be a two-part substrate and include a lid plate 21 and a through-hole plate 23. The substrate support 22 can be made of or include a non-porous, gas-permeable material and can also include a plurality of alignment and sealing pads 36. The sealing pads 36 can be in the shape of a cylinder, a ring, or any other shape, for example, that complements and corresponds to the shape of a bottom portion or opening of the sample-containment region (not shown in FIG. 1) to be sealed. The sealing pads 36 can include or be made of the same material as the substrate support 22, or can include a different material. According to various embodiments, both the substrate support and the pads include a non-porous, gas-permeable material. The substrate support 22 can be operatively aligned with the substrate 20 and the plurality of through-holes 26 can align with and be sealed by the pads 36, such as shown, to form respective sample-containment regions. To assist in alignment, crosshairs can be formed respectively on the substrate support 22, the substrate 20, and the lid plate 21, or other alignment devices can be used, for example, one or more recess or through hole and one or more corresponding alignment pin. For example, crosshairs can be molded in, etched in, or marked on, the respective components.

The microfluidic device can have dimensions of any length, width, and depth. For example, the length can be from about one inch to about 10 inches, or from about four inches to about six inches. The width can be from about 0.5 inch to about eight inches, or from about two inches to about four inches. The depth can be from about 0.1 millimeter (mm) to about 50 mm, or from about 1.0 mm to about 20 mm. Each layer or element of the microfluidic device can have the aforementioned length and width dimensions, for example, each of the lid plate, substrate, and substrate support. According to various embodiments, the microfluidic device can have an outer peripheral shape or footprint of a standard microtiter plate, that is, a length of about five inches and a width of about 3.25 inches.

The thickness or depth of the entire microfluidic device can be from about 0.1 mm to about 50 mm as mentioned above, and each element of the microfluidic device can independently constitute a major portion of that thickness. The thickness of the substrate and the substrate support can be the same or different and each can independently be from about 0.01 mm to about 50 mm or from about 1.0 mm to about 10 mm. The lid plate or a cover layer can be included that has a thickness of, for example, from about 0.0001 mm to about 10 mm, or from about 0.1 mm to about 1.0 mm.

FIG. 2 is an enlarged cross-sectional side view of a microfluidic device 10 similar to that shown in FIG. 1, but including a greater number of sample-containment regions 16 and shown after assembly. FIG. 2 shows the microfluidic device 10 hinged shut such that the plurality of sealing pads 36 seals the plurality of through-holes 26. The microfluidic device 10 can include a plurality of input openings 12, a plurality of valves 15, a plurality of loading input channels 14, a plurality of outlet openings 18, and a plurality of frangible seals 11. The input channels 14 can be in fluid communication with respective sample-containment regions 16 that can include through-holes 26. The sample-containment regions 16 can be in fluid communication with respective outlet openings 18. Each outlet opening 18 can be sealed with a respective non-porous, gas-permeable material device in the form of a sealing plug 30. The non-porous, gas-permeable sealing plugs 30 can include, for example, a polysiloxane material, or a polydimethylsiloxane material. The substrate support 22 can be made of or include a non-porous, gas-permeable material.

FIG. 2 also shows an injection device 17 that can be inserted into one of the plurality of input openings 12 to load a liquid sample through a respective valve 15 and into a respective loading input channel 14. The injection device 17 can include, for example, a pipette or a micropipette.

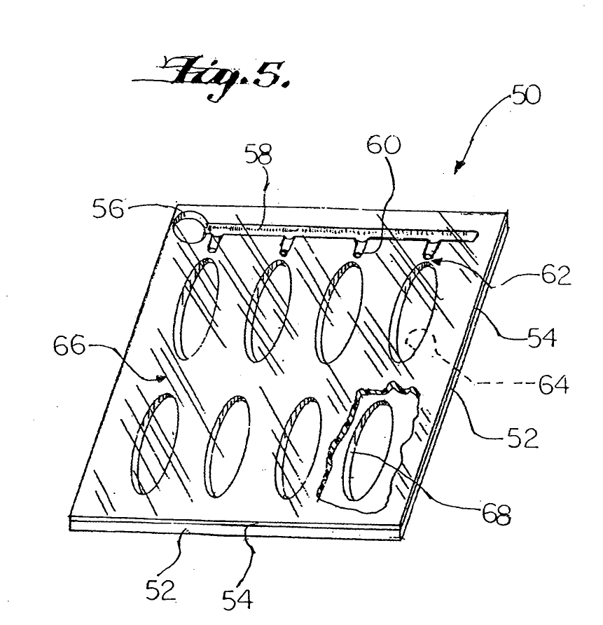

FIG. 3 is an exploded view of an assembly 100 including a substrate support 22 that includes a plurality of pads 36 formed thereon, and a mask 32 including a plurality of through-holes 34 formed therein and used to form the pads 36. The mask 32 can include a plurality of through-holes 34. The mask can be used to form the pads 36 on the substrate support 22 in a spaced and aligned arrangement, for example, in the arrangement shown. According to various embodiments, the pads 36 can include a hydrophilic material. According to various embodiments, the substrate support 22 can instead or additionally include, for example, a hydrophobic material. The pads 36 can be aligned with corresponding through-holes 26 on the substrate 20 as shown in FIG. 2 to ensure accurate alignment of the substrate 20 on the substrate support 22 when the substrate 20 and substrate support 22 are hinged together. The mask 32 can be placed over the substrate support 22 during a preparation method, for example, to enable deposition of pad material on the substrate support 22.

FIG. 4 is an enlarged view of a portion of FIG. 3 showing one of the pads 36 on the substrate support 22. The pads 36 can be composed of hydrophilic material deposited on the substrate support 22 by a preparation method wherein the hydrophilic material is directed toward the top face of the mask 32 and is caused to pass through the through-holes 34 and become deposited on the top surface of the substrate support 22. Methods of applying and depositing the pad material onto the substrate support 22 whether or not through the mask 32 can include electro-spark deposition (ESD) techniques, chemical etching, plasma deposition, chemical vapor deposition, screen printing, injection molding, insert molding, casting, physical placement, adhesive bonding of discrete elements, and other methods known to those of skill in the art. Various other plastic molding techniques known to those of skill in the art can be used to prepare the substrate support and pads. According to various embodiments, the pads can be spotted or pre-spotted with any of probes, primers, analytes, controls, dyes, nucleic acid sequences, or other reactants or chemicals, for example, components useful in a nucleic acid sequencing or amplification reaction, any of which can be dried down after application in a wet form, or applied by a different technique. Each pad 36 can independently be spotted or pre-spotted with different materials, for example, with a different probe and/or primer, or with the same materials as used to spot or pre-spot one or more other pads 36.

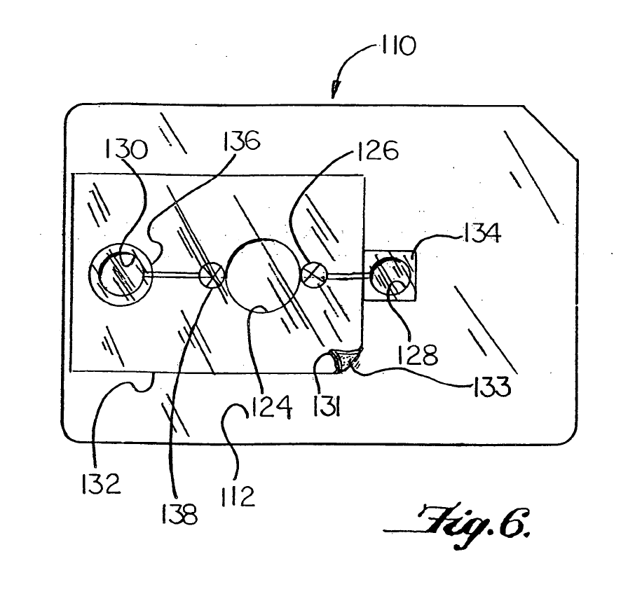

FIG. 5 is a perspective view of a microfluidic device 50 including a substrate 52 and a non-porous, gas-permeable material cover layer 54. The substrate 52 includes an input chamber 56, a loading channel 58, a plurality of branch channels 60, and a plurality of valves 62. The plurality of valves 62 can be opened to form respective input openings (not shown), one for each sample-containment region 64. For the plurality of sample-containment regions 64, a respective plurality of valves 66 can be opened to form respective fluid communications between the plurality of sample-containment regions 64 and a respective plurality of second sample-containment regions 68. Both plurality of valves 62 and 66 can be closed subsequent to being opened. As can be seen in FIG. 5, the sample-containment regions can be in the form of sample-containment wells or other recesses in or through the substrate 52.

According to various embodiments, the non-porous, gas-permeable material sealing cover layer can be secured to the substrate 52 by way of an adhesive, by heat bonding, by ultrasonic bonding, or by other application methods known to those of skill in the art. The sealing cover layer can be hermetically sealed to an upper surface of the substrate 52. The sealing cover layer can be in such intimate contact with the substrate that little, if any, leaking of an aqueous sample occurs between the sealing cover layer and the substrate, for example, under a pressure of 50 psi water at 95° C. After the valves 62 are opened, liquid sample from the loading channels can be forced into sample-containment regions 64 by pressure, vacuum, gravitational force, centripetal force, or the like. Likewise, sample portions from sample-containment regions 64 can be transferred by pressure, vacuum, gravitational force, centripetal force, or the like, into respective sample-containment regions 68 after respective valves 66 are opened. Any number of sample-containment regions, valves, channels, purification columns, flow splitters, or other microfluidic device features, can be included in the microfluidic device. The microfluidic device features can range in size, for example, from about 1 micron to about 500 microns. Gas contained in, or generated in, sample-containment regions 64, 68, or 64 and 68, can be vented by molecular diffusion through the sealing cover layer 54. The ready diffusion of gas through the cover layer 54 can be provided by forming the sealing cover layer 54 of a polysiloxane material, for example, polydimethylsiloxane. The sealing cover layer 54 can have an exemplary thickness of from about 0.001 inch to about 0.1 inch, for example, from about 0.003 inch to about 0.05 inch. Before, during, or after use, the microfluidic device can be further coated, sealed by, or covered by, or can be provided initially coated, sealed, or covered by, a gas-impermeable layer, for example, a non-porous aluminum film layer, a polyolefin film layer, or a polytetrafluoroethylene layer. The gas-impermeable layer can be capable of preventing evaporation, or other loss, or contamination, of a sample within the sample-containment region.

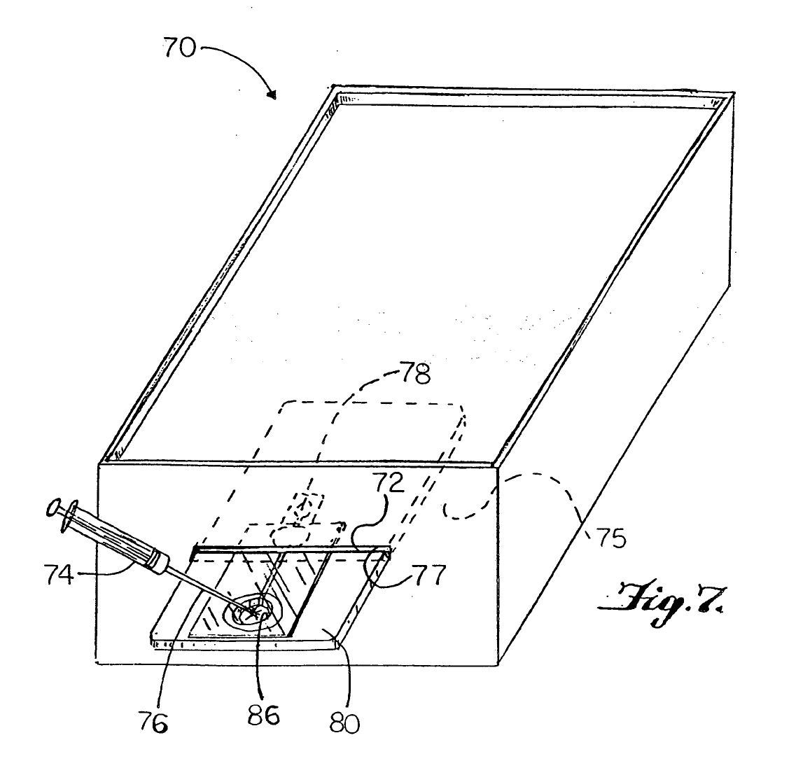

FIG. 6 is a plan view of an embodiment of a microfluidic device 110 on a substrate 112 that includes an inlet opening 130 covered by a pierceable material 136, a sample-containment region 124, and a venting region 128 that is covered by a gas-permeable material 134. Fluid communication between the inlet chamber 130, the sample-containment region 124, and the venting region 128 can be controlled by valves 126. The sample-containment region 124 can be covered by a non-gas-permeable cover material 132. The non-gas-permeable cover layer 132 can include the pierceable material 136 and can be located with respect to inlet chamber 130. The non-gas-permeable cover layer 132 can include, for example, a film layer 131 and an adhesive layer 133. A sample can be delivered to the inlet chamber 130 by, for example, a syringe (not shown in FIG. 6) and the valve 138 can be open to permit the sample to flow into the sample-containment region 124. The valve 126 can be opened as desired to permit gas venting through the venting region 128. The non-gas-permeable cover layer 132 can include, for example, glass, or any other gas-impermeable material that is, a material that has a permeability coefficient related to O₂ of 8×10¹⁵ or lower at 35° C.

According to various embodiments, the process of loading a sample into the microfluidic device can be assisted by a vacuum-assisted method wherein a vacuum can be drawn on an exit port or outlet of the microfluidic device, to load a sample. The sample can be loaded into the inlet port and a sample can be drawn into the sample-containment region by application of a vacuum to an appropriate region or regions of the microfluidic device.

FIG. 7 is a perspective view of an embodiment of a vacuum loading apparatus 70 and a microfluidic device 80 that includes an inlet chamber 86 and a non-porous gas-permeable material cover layer. The microfluidic device 80 is shown positioned in the vacuum loading apparatus. The vacuum loading apparatus is provided with an interior 75 and a device opening 77. The inlet chamber 86 of the microfluidic device can be operatively located outside of the vacuum chamber 70. A sealing curtain 72 can seal around the microfluidic device 80 in the vicinity of the device opening 77 to provide a hermetic seal. A vacuum can be applied to the microfluidic device 80 from the interior 75 of the vacuum chamber 70 and a sample can be delivered to the inlet chamber 86 by, for example, a syringe body 74 and cannula 76. The sample can be drawn by vacuum into the microfluidic device 80 and into the sample-containment region 78.

According to various embodiments, the sample-containment region can include at least one well, recess, depression, indentation, through-hole, or reservoir capable of containing a sample, and a non-porous, gas-permeable material sealing device for sealing the sample-containment region. The non-porous, gas-permeable material sealing device can include at least one sidewall. The non-porous, gas-permeable material can be, for example, a polysiloxane material. Fluid communication to and from the sample-containment region can be provided through two valves and an exit port can be provided in liquid fluid communication with the sample-containment region when one of the valves is opened.

The non-porous, gas-permeable material of the sealing device, whether in the form of a plug or a cover layer such as a film, sheet, or strip, can include at least one member selected from polysiloxane materials, polydimethylsiloxane materials, polydiethylsiloxane materials, polydipropylsiloxane materials, polydibutylsiloxane materials, polydiphenylsiloxane materials, and other polydialkylsiloxane or polyalkylphenylsiloxane materials. The sealing device can be in the form of a sealing strip that fits into a groove on a surface of the microfluidic device. The polysiloxane can be the reaction product of an uncrosslinked reactive polysiloxane monomer and from about 0.01 weight percent to about 50 weight percent polysiloxane crosslinker, for example, from about 0.1 weight percent to about 25 weight percent or from about 0.5 weight percent to about 10 weight percent polysiloxane crosslinker.

The non-porous, gas-permeable material, whether in the form of a plug or a cover layer, can include a polysiloxane material, a polyalkylsiloxane material, a polydialkylsiloxane material, a polyalkylalkylsiloxane material, a polyalklyarylsiloxane material, a polyarylsiloxane material, a polydiarylsiloxane material, a polyarylarylsiloxane material, a polycycloalkylsiloxane material, a polydicycloalkylsiloxane material, and combinations thereof. According to various embodiments, the polysiloxane material can include, for example, RTV 615, a polydimethylsiloxane material available from GE Silicones of Waterford, N.Y. The polysiloxane can be formed of a two-part silicone, for example RTV 615.

According to various embodiments, a method is provided whereby the liquid fluid communication between the sample-containment region and an input opening is sealed off after a sample is provided in the sample-containment region. The liquid fluid communication between the sample-containment regions and respective input openings can be sealed off by respective valves located in respective loading input channels between the sample-containment regions and the input openings. The non-porous, gas-permeable material sealing device can seal off a set or group of sample-containment regions from liquid fluid communication with any other set or group of sample-containment regions present in the device.

According to various embodiments, the microfluidic device can have at least two, at least four, at least 48, at least 96, at least 1,000, at least 10,000, at least 30,000, or at least 100,000 sample-containment regions capable of receiving and retaining a sample.

According to various embodiments, the sample-containment region of the device can be sealed all around except at a sidewall area defined by the non-porous, gas-permeable material sealing device, and except at an area adjacent an input opening to the sample-containment region. The liquid fluid communication between the input opening and the sample-containment region can be sealed off after a sample is disposed in the sample-containment region. According to various embodiments, the liquid fluid communication between each of the various regions or microfluidic device features included in the microfluidic device can be established or discontinued by opening or closing, for example, a valve.

According to various embodiments, the non-porous, gas-permeable material sealing device can be positioned in a location that avoids contact or interaction with a sample. Such undesirable interactions can include quenching of fluorescence during or resulting from a PCR reaction. The sample can be positioned so as not to contact the non-porous, gas-permeable material sealing device while the sample is in, for example, a liquid form, or a solid form.

According to various embodiments, the non-porous, gas-permeable material sealing device can be located in an exit port that can be sealed off from liquid fluid communication with a sample-containment region, by a valve. According to various embodiments, the sample-containment region can be formed between the above mentioned valve and at least one or more other valves.

According to various embodiments, a method for venting a gas from a microfluidic device is provided. The method can include loading a liquid into a microfluidic device and venting a gas from the microfluidic device through a non-porous, gas-permeable material sealing device. The non-porous, gas-permeable material sealing device can be, for example, in the form of a plug or a cover layer such as a film, sheet, or strip. The venting of gas through the non-porous, gas-permeable sealing cover layer can occur solely by molecular diffusion of the gas through the non-porous, gas-permeable material. According to various embodiments, the method can further include applying an impermeable membrane to the non-porous, gas-permeable sealing device after gas has been vented through the non-porous, gas-permeable material sealing device.

According to various embodiments, a method is provided for filling or loading a microfluidic device with a sample. The loading can be accomplished free of a vacuum-assist or with a vacuum drawn on the outlet or exit port. The sample can include one or more liquids, one or more solids, one or more gases, or a combination thereof. The sample can include a solid that is powdered, pelleted, particulate, granulated, slurried, or the like. The method can include venting a gas formed as a result of a reaction, for example, a reaction within the sample-containment region.

According to various embodiments, a method is provided for loading a microfluidic device, the method can include providing a microfluidic device including a plurality of sample-containment regions, loading one or more of the plurality of sample-containment regions with a sample that may or may not contain an analyte of interest, and sealing the selected sample-containment regions by placing a non-porous, gas-permeable material cover layer on the microfluidic device. The sealing can occur before or after loading the sample. Loading can include, for example, filling the sample-containment regions. According to various embodiments, a respective selection of the plurality of the sample-containment regions of the microfluidic device can be loaded with appropriately and/or respectively labeled nucleic acid sequence probes, nucleic acid sequence primers, or both. The loading can be accomplished prior to, and/or subsequent to, the introduction of the sample and/or the sealing of the device.

The loading of the microfluidic devices can be performed on an assembly line. A non-gas-permeable cover layer can be applied to the non-porous, gas-permeable material sealing device and can be used to eliminate sample loss due to evaporation and/or to avoid contamination. Evaporation could otherwise result due to heating, for example, in a thermocycled nucleic acid amplification reaction such as a polymerase chain reaction (“PCR”). Evaporation can also occur from storing a sample in the microfluidic device for a long period of time, for example, for more than one hour. The non-gas-permeable cover layer can also prevent contamination of a sample once retained in the microfluidic device.

According to various embodiments, the microfluidic device can include one or more of a block, substrate, through hole plate, well plate, cover, or other element that can be made of, or include, a thermally conductive polymeric material. Examples of thermally conductive polymeric materials that can be used include those polymeric materials having a thermal conductivity value of about 0.5 Watts per meter ° Kelvin (W/mK) or greater. Such polymeric materials include, for example, the filled polypropylene RTP 199×91020 A Z available from RTP Company, Winona, Minn., and the filled polypropylene Coly Poly E1201 available from Cool Polymers, of Warwick, R.I.

According to various embodiments, a method of hydrating the microfluidic device can be provided. The hydration of the non-porous, gas-permeable material sealing device can eliminate or reduce evaporation of a sample from the microfluidic device at elevated temperatures. Methods and systems of hydrating microfluidic devices are known to those of skill in the art. Hydration parts and channels can be provided in the microfluidic device and arrayed to supply a hydrating material to one or more components of the microfluidic device, for example, to hydrate the non-porous, gas-permeable sealing device.

According to various embodiments, a method is provided for making a microfluidic device that includes a non-porous, gas-permeable material sealing device. The method can include positioning a mask including through-holes on a microfluidic device support and depositing or otherwise applying a first material to the masked side of the resulting assembly, thereby forming pads or spots on the microfluidic device support. The first material can be applied by gas plasma treatment, electro-spark deposition (ESD), chemical vapor deposition, chemical etching, machining, screen printing, adhesion, or by other means known to those of skill in the art. These application techniques can also be used to alter the hydrophobicity of the materials. The mask can then be removed. Upon removing the mask, pads or spots of applied material remain on the underlying substrate support surface. The pads or spots can then be treated, or they can be treated before the mask is removed. According to various embodiments, the substrate support can be formed by a plastic molding or plastic forming process. The substrate can be manufactured by one or more processes including stamping, punching, micromachining, molding, casting, or by any other suitable process known to those of skill in the art.

The method can further include aligning the substrate with the pads or spots on the substrate support. The microfluidic device can include through-holes which align with the pads on the substrate support. The microfluidic device can then be positioned on the substrate support and fixedly attached thereto. The pads can include the same or different material or materials as the substrate support. According to various embodiments, the pads can be formed of hydrophilic material while the substrate support can be formed of hydrophobic material. According to various embodiments, the substrate support can be flexibly hinged to the microfluidic device to align the device with the substrate support.

Further microfluidic devices, substrates, covers, input openings, input channels, outlet openings, pathways, valves, reagents, flow restrictors, microfluidic device manufacturing methods, and methods of use that can be used according to various embodiments, are described in U.S. Patent Applications Nos. 10/336,706, 10/336,274, and 10/336,330, all filed Jan. 3, 2003, and Ser. No. 10/625,449, filed Jul. 23, 2003, and all of which are incorporated herein in their entireties by reference.

Microfluidic devices and systems as described herein can facilitate the venting of gas from the microfluidic device and can subsequently reduce back pressure during liquid sample loading. The features and methods described herein can also be used with existing microfluidic device technologies and designs.

Those skilled in the art can appreciate from the foregoing description that the broad teachings of the present application can be implemented in a variety of forms. Therefore, while these teachings have been described in connection with particular embodiments and examples thereof, the true scope of the present teachings should not be so limited.

1. A microfluidic device comprising:
at least one sample-containment region capable of containing a sample;
at least one non-porous, gas-permeable sample sealing plug at least partially defining the at least one sample-containment region, and comprising a non-porous, gas-permeable material having a permeability coefficient at about 35° C. relative to O₂ of at least about 8×10¹⁵; and
an input opening in fluid communication with the sample-containment region.

2. The microfluidic device of claim 1, wherein the sample-containment region further comprises at least one sidewall that is gas-permeable and impermeable to water at 50 psi and at a temperature from about 25° C. to about 95° C.

3. The microfluidic device of claim 1, wherein the non-porous, gas-permeable material comprises a polysiloxane material.

4. The microfluidic device of claim 1, wherein the non-porous, gas-permeable material comprises at least one material selected from polydimethylsiloxane materials, polydiethylsiloxane materials, polydiphenylsiloxane materials, polymethylethylsiloxane materials, polymethylphenylsiloxane materials, and combinations thereof.

5. The microfluidic device of claim 1, wherein the non-porous, gas-permeable material comprises a polydialkylsiloxane material.

6. The microfluidic device of claim 1, wherein the non-porous, gas-permeable material comprises a polydimethylsiloxane material.

7. The microfluidic device of claim 1, wherein the non-porous, gas-permeable material comprises the reaction product of an uncrosslinked reactive polysiloxane monomer and from about 0.01 percent by weight to about 50 percent by weight of a polysiloxane crosslinker.

8. The microfluidic device of claim 1, wherein:
the fluid communication comprises a channel between the input opening and the sample-containment region; and
the channel includes a valve.

9. The microfluidic device of claim 8, wherein the valve is in a closed state and the fluid communication through the channel is interrupted.

10. The microfluidic device of claim 1, wherein the at least one sample-containment region comprises a plurality of sample-containment regions and the at least one non-porous, gas-permeable sample sealing plug comprises a plurality of non-porous, gas-permeable sample sealing plugs.

11. The microfluidic device of claim 1, wherein the at least one sample-containment region comprises at least four sample-containment regions and the at least one non-porous, gas-permeable sealing plug comprises at least four non-porous, gas-permeable sealing plugs that respectively at least partially define the at least four sample-containment regions.

12. The microfluidic device of claim 1, wherein the at least one sample-containment region comprises at least 96 sample-containment regions and the at least one non-porous, gas-permeable sealing plug comprises at least 96 non-porous, gas-permeable material sealing plugs that respectively at least partially define the at least 96 sample-containment regions.

13. The microfluidic device of claim 1, wherein the at least one sample-containment region comprises at least 1,000 sample-containment regions and the at least one non-porous, gas-permeable sealing plug comprises at least 1,000 non-porous, gas-permeable material sealing plugs that respectively at least partially define the at least 1,000 sample-containment regions.

14. The microfluidic device of claim 1, wherein the at least one sample-containment region comprises at least 30,000 sample-containment regions and the at least one non-porous, gas-permeable sealing plug comprises at least 30,000 non-porous, gas-permeable material sealing plugs that respectively at least partially define the at least 30,000 sample-containment regions.

15. The microfluidic device of claim 1, wherein the at least one sample-containment region contains a sample disposed therein.

16. The microfluidic device of claim 1, wherein the sample-containment region contains a dried sample.

17. The microfluidic device of claim 1, wherein the sample-containment region further comprises at least one of a nucleic acid sequence probe or nucleic acid sequence primer disposed therein.

18. The microfluidic device of claim 17, wherein the at least one nucleic acid sequence probe or nucleic acid sequence primer is in a dried form.

19. The microfluidic device of claim 1, wherein the at least one sample-containment region comprises a plurality of sample-containment regions arranged in an array.

20. The microfluidic device of claim 19, wherein a selected plurality of the sample-containment regions contain one of a nucleic acid sequence probe, a nucleic acid sequence primer, or a sample containing an analyte of interest.

21. The microfluidic device of claim 19, wherein a selected plurality of the sample-containment regions containing a sample, a nucleic acid sequence probe, or a nucleic acid sequence primer are arranged in one or more of a selected row or a selected column of the array.

22. A microfluidic device comprising:
at least one sample-containment region;
a non-porous, gas-permeable sample sealing cover layer at least partially defining the at least one sample-containment region and comprising a non-porous, gas-permeable material having a permeability coefficient at about 35° C. relative to O₂ of at least about 8×10¹⁵; and
an input opening in fluid communication with the at least one sample-containment region.

23. The microfluidic device of claim 22, wherein the at least one sample-containment region comprises at least one sidewall that is gas-permeable and impermeable to water at 50 psi and at a temperature from about 25° C. to about 95° C.

24. The microfluidic device of claim 22, wherein the non-porous, gas-permeable material comprises a polysiloxane material.

25. The microfluidic device of claim 22, wherein the non-porous, gas-permeable material comprises at least one member selected from polydimethylsiloxane materials, polydiethylsiloxane materials, polydiphenylsiloxane materials, polymethylethylsiloxane materials, polymethylphenylsiloxane materials, and combinations thereof.

26. The microfluidic device of claim 22, wherein the non-porous, gas-permeable material comprises a polydialkylsiloxane material.

27. The microfluidic device of claim 22, wherein the non-porous, gas-permeable material comprises a polydimethylsiloxane material.

28. The microfluidic device of claim 22, wherein:
the fluid communication comprises a channel between the input opening and the at least one sample-containment region; and
the channel includes a valve.

29. The microfluidic device of claim 28, wherein the valve is in a closed state and the fluid communication through the channel is interrupted.

30. The microfluidic device of claim 22, wherein the at least one sample-containment region comprises a plurality of sample-containment regions and the non-porous, gas-permeable sealing cover layer at least partially defines the plurality of sample-containment regions.

31. The microfluidic device of claim 22, wherein the at least one sample-containment region comprises a plurality of sample-containment regions and the non-porous, gas-permeable sealing cover layer interrupts fluid communication from one of the plurality of sample-containment regions to the others of the plurality of sample-containment regions.

32. The microfluidic device of claim 22, wherein the at least one sample-containment region comprises at least four sample-containment regions and the at least one non-porous, gas-permeable sealing cover layer comprises at least four non-porous, gas-permeable material sealing cover layers that respectively at least partially define the at least four sample-containment regions.

33. The microfluidic device of claim 22, wherein the at least one sample-containment region comprises at least 96 sample-containment regions and the at least one non-porous, gas-permeable sealing cover layer comprises at least 96 non-porous, gas-permeable material sealing cover layers that respectively at least partially define the at least 96 sample-containment regions.

34. The microfluidic device of claim 22, wherein the at least one sample-containment region comprises at least 1,000 sample-containment regions and the at least one non-porous, gas-permeable sealing cover layer comprises at least 1,000 non-porous, gas-permeable material sealing cover layers that respectively at least partially define the at least 1,000 sample-containment regions.

35. The microfluidic device of claim 22, wherein the at least one sample-containment region comprises at least 30,000 sample-containment regions and the at least one non-porous, gas-permeable sealing cover layer comprises at least 30,000 non-porous, gas-permeable material sealing cover layers that respectively at least partially define the at least 30,000 sample-containment regions.

36. The microfluidic device of claim 22, wherein the sealing cover layer comprises a sealing strip.

37. A microfluidic device comprising:
at least one sample-containment region;
at least one non-gas-permeable material at least partially defining the at least one sample-containment region;
at least one venting region in fluid communication with the at least one sample-containment region; and
at least one non-porous, gas-permeable sealing device at least partially defining the at least one venting region and comprising a non-porous, gas-permeable material having a permeability coefficient relative to O₂ at about 35° C. of at least about 8×10¹⁵.

38. The microfluidic device of claim 37, wherein the gas-permeable sealing device comprises a cover layer.

39. The microfluidic devices of claim 37, wherein the gas-permeable sealing device comprises a sealing plug.

40. The microfluidic device of claim 37, wherein the at least one venting region further comprises at least one sidewall that is gas-permeable and impermeable to water at a water pressure of 50 psi and at a temperature from about 25° C. to about 95° C.

41. The microfluidic device of claim 37, wherein the non-porous, gas-permeable material comprises a polysiloxane material.

42. The microfluidic device of claim 37, wherein the non-porous, gas-permeable material comprises at least one material selected from polydimethylsiloxane materials, polydiethylsiloxane materials, polydiphenylsiloxane materials, polymethylethylsiloxane materials, polymethylphenylsiloxane materials, and combinations thereof.

43. The microfluidic device of claim 37, wherein the non-porous, gas-permeable material comprises a polydialkylsiloxane material.

44. The microfluidic device of claim 37, wherein the non-porous, gas-permeable material comprises a polydimethylsiloxane material.

45. The microfluidic device of claim 37, wherein the non-porous, gas-permeable material comprises the reaction product of an uncrosslinked reactive polysiloxane monomer and from about 0.01 percent by weight to about 50 percent by weight of a polysiloxane crosslinker.

46. The microfluidic device of claim 37, wherein:
the fluid communication comprises a channel between the venting region and the sample-containment region; and
the channel includes a valve.

47. The microfluidic device of claim 46, wherein the valve is in a closed state and the fluid communication through the channel is interrupted.

48. The microfluidic device of claim 37, wherein the at least one venting region comprises an exit port.

49. The microfluidic device of claim 37, wherein the at least one non-porous, gas-permeable sealing plug comprises a plurality of non-porous, gas-permeable sealing plugs.

50. The microfluidic device of claim 49, wherein each one of the plurality of one non-porous, gas-permeable sealing plugs respectively partially defines at least one venting region of a plurality of venting regions.

51. The microfluidic device of claim 37, wherein the at least one venting region comprises a plurality of venting regions and the at least one non-porous, gas-permeable sealing plug comprises a plurality of non-porous, gas-permeable sealing plugs.

52. The microfluidic device of claim 37, wherein the at least one venting region comprises at least four venting regions and the at least one non-porous, gas-permeable sealing plug comprises at least four non-porous, gas-permeable sealing plugs that respectively at least partially define the at least four venting regions.

53. The microfluidic device of claim 37, wherein the at least one venting region comprises at least 96 venting regions and the at least one non-porous, gas-permeable sealing plug comprises at least 96 non-porous, gas-permeable material sealing plugs that respectively at least partially define the at least 96 venting regions.

54. The microfluidic device of claim 37, wherein the at least one venting region comprises at least 1,000 venting regions and the at least one non-porous, gas-permeable sealing plug comprises at least 1,000 non-porous, gas-permeable material sealing plugs that respectively at least partially define the at least 1,000 venting regions.

55. The microfluidic device of claim 37, wherein the at least one venting region comprises at least 30,000 venting regions and the at least one non-porous, gas-permeable sealing plug comprises at least 30,000 non-porous, gas-permeable material sealing plugs that respectively at least partially define the at least 30,000 venting regions.

56. The microfluidic device of claim 37, wherein the at least one sample-containment region comprises a plurality of sample-containment regions and the at least one non-gas-permeable cover layer comprises a plurality of non-gas-permeable cover layers that respectively at least partially define the plurality of sample-containment regions.

57. The microfluidic device of claim 37, wherein the at least one sample-containment region comprises at least four sample-containment regions and the at least one non-gas-permeable cover layer comprises at least four non-gas-permeable cover layers that respectively at least partially define the at least four sample-containment regions.

58. The microfluidic device of claim 37, wherein the at least one sample-containment region comprises at least 96 sample-containment regions and the at least one non-gas-permeable cover layer comprises at least 96 non-gas-permeable cover layers that respectively at least partially define the at least 96 sample-containment regions.

59. The microfluidic device of claim 37, wherein the at least one sample-containment region comprises at least 1,000 sample-containment regions and the at least one non-gas-permeable cover layer comprises at least 1,000 non-gas-permeable cover layers that respectively at least partially define the at least 1,000 sample-containment regions.

60. The microfluidic device of claim 37, wherein the at least one sample-containment region comprises at least 30,000 sample-containment regions and the at least one non-gas-permeable cover layer comprises at least 30,000 non-gas-permeable cover layers that respectively at least partially define the at least 30,000 sample-containment regions.

61. A method for venting a gas from a microfluidic device comprising:
providing a microfluidic device, the microfluidic device comprising;
at least one sample-containment region capable of containing a sample;
at least one non-porous, gas-permeable sample sealing plug at least partially defining the at least one sample-containment region, and comprising a non-porous, gas-permeable material;
an input opening in fluid communication with the sample-containment region;
loading a liquid into the microfluidic device; and
venting a gas from the microfluidic device through the at least one non-porous, gas-permeable sample sealing plug.

62. The method of claim 61, wherein the non-porous, gas-permeable material comprises a material having a permeability coefficient at about 35° C. relative to O₂ of at least about 8×10¹⁵.

63. The method of claim 61, wherein the non-porous, gas-permeable material comprises a polysiloxane material.

64. The method of claim 61, wherein the non-porous, gas-permeable material comprises at least one member selected from polydimethylsiloxane materials, polydiethylsiloxane materials, polydiphenylsiloxane materials, polymethylethylsiloxane materials, polymethylphenylsiloxane materials, and combinations thereof.

65. The method of claim 61, wherein the non-porous, gas-permeable material comprises a polydialkylsiloxane material.

66. The method of claim 61, wherein the non-porous, gas-permeable material comprises a polydimethylsiloxane material.

67. The method of claim 61 further comprising applying a gas-impermeable membrane to the at least one non-porous, gas-permeable sample sealing plug.

68. The method of claim 61, wherein the microfluidic device includes a channel in fluid communication with the sample-containment region, and the method further includes interrupting fluid communication through the channel.

69. A method for venting a gas from a microfluidic device comprising:
providing a microfluidic device, the microfluidic device comprising;
at least one sample-containment region capable of containing a sample;
at least one non-porous, gas-permeable sample sealing cover layer at least partially defining the at least one sample-containment region, and comprising a non-porous, gas-permeable material;
an input opening in fluid communication with the sample-containment region;
loading a liquid into the microfluidic device; and
venting a gas from the microfluidic device through the at least one non-porous, gas-permeable sample sealing cover layer.

70. The method of claim 69, wherein the non-porous, gas-permeable material comprises a material having a permeability coefficient at about 35° C. relative to O₂ of at least about 8×10¹⁵.

71. The method of claim 69, wherein the non-porous, gas-permeable material comprises polysiloxane material.

72. The method of claim 69, wherein the non-porous, gas-permeable material comprises at least one member selected from polydimethylsiloxane materials, polydiethylsiloxane materials, polydiphenylsiloxane materials, polymethylethylsiloxane materials, polymethylphenylsiloxane materials, and combinations thereof.

73. The method of claim 69, wherein the non-porous, gas-permeable material comprises a polydialkylsiloxane material.

74. The method of claim 69, wherein the non-porous, gas-permeable material comprises a polydimethylsiloxane material.

75. The method of claim 69, further comprising applying a gas-impermeable membrane to the at least one non-porous, gas-permeable sample sealing cover layer.

76. The method of claim 69, wherein the microfluidic device includes a channel in fluid communication with the sample-containment region, and the method further includes interrupting fluid communication through the channel.

77. A method comprising:
providing a microfluidic device including a plurality of sample-containment regions;
loading the plurality of sample-containment regions with a sample to form loaded sample-containment regions; and
sealing the loaded sample-containment regions with a non-porous, gas-permeable material cover layer.

78. The method of claim 77, further comprising:
loading a nucleic acid sequence probe or a nucleic acid sequence primer into selected sample-containment regions.

79. The method of claim 78, wherein the nucleic acid sequence probe or the nucleic acid sequence primer is loaded into the loaded sample-containment regions.

80. The method of claim 78, wherein the nucleic acid sequence probe or the nucleic acid sequence primer is loaded prior to loading the plurality of sample-containment regions with the sample.
